(19) United States
(12) Patent Application Publication
(10) Pub. No.: US 2012/0312754 A1
(43) Pub. Date: Dec. 13, 2012

(54) CONTROL OF SUBSEA CYCLONE

(75) Inventors: Klas Goran Eriksson, Asker (NO); Geir Inge Olsen, Oslo (NO); Steinar Oyulvstad, Slependen (NO)

(73) Assignee: Aker Subsea AS, Lysaker (NO)

(21) Appl. No.: 13/518,461

(22) PCT Filed: Dec. 20, 2010
(86) PCT No.: PCT/NO2010/000478

§ 371 Date: Aug. 30, 2012

(30) Foreign Application Priority Data
Dec. 29, 2009 (NO) 20093600

(51) Int. Cl. E21B 43/36; G01N 33/28; B01D 19/00; C02F 1/38; B04C 11/00

(52) U.S. Cl. 210/739; 210/96.1; 96/156; 95/1; 73/61.43

(57) ABSTRACT

System and method for control of a subsea located cyclone for separating oil from water. The cyclone is arranged to receive water with oil contents through an inlet line, the oil is separated from the water and delivered through an oil outlet to an oil outlet line, and the water is delivered through a water outlet to a water outlet line. The system is comprising a control valve in the oil outlet or oil outlet line from the cyclone, a first differential pressure transducer arranged between the inlet line and the oil outlet from the cyclone, and a second differential pressure transducer arranged between the inlet line and the water outlet from the cyclone. The system is distinguished in that a sensor for measuring oil contents is arranged in the water outlet or water outlet line, and via a control means said sensor is operatively connected to the control valve.

### FIELD OF THE INVENTION

The present invention relates to cyclones. More specifically, the present invention relates to control of subsea located cyclones for separating oil from a mixed flow of oil and water.

### BACKGROUND OF THE INVENTION AND PRIOR ART

Cyclone separators are well known equipment using rotational effects, in addition to gravity, in order to separate fluids and/or solids. Cyclones for separating liquids are often termed hydrocyclones. Hydrocyclones have one inlet and two exits, one exit for the heavier phase at the apex of a conical section and one exit for the lighter phase at the opposite end, at the end of a typically cylindrical section. A hydrocyclone used for separating oil from a flow mainly comprising water can be called a reverse type hydrocyclone, since the lighter phase oil is removed from the heavier phase water. The subject matter of the present invention is not the cyclones per se, but control of cyclones, for which reason cyclones will not be described in further detail.

The control of cyclones is typically based on models of the separation effect and how it relates to parameters like pressure and flow. For cyclones on industrial sites and offshore platforms, samples can be taken or measurements made of the flow in order to verify that the control of the operation is as intended. For cyclones located subsea, sometimes at many hundred meters of depth and tens or hundreds of kilometers away from shore or surface installations, control of the operation is difficult. For subsea cyclones separating oil from water, the water can be injected into the reservoir and the oil can be transported together with oil from preceding separation equipment. Watery oil must be processed later, at a cost, whilst oily water may cause problems in the reservoir and oily injection water means that some of the valuable oil is re-injected into the reservoir once it has been produced.

Typically injection water should have an oil content of 100 ppm or less. Excessive oil content in injection water can damage the injectivity of the well by clogging the pores in the well formation. Such an effect is often reversible, i.e. by injecting cleaner water the pores may be flushed clean, and injectivity restored. The damage is from dispersed liquid droplets, i.e. dissolved hydrocarbons have little or no effect on injectivity.

Excessive solids content in the injection water can likewise damage the injectivity of a well by clogging the pores in the well formation. However such a damage to the well is more often irreversible, and may require a costly well intervention to restore injectivity.

It is thus desirable to be able to measure the amount of liquid droplets and solids particles simultaneously.

Currently there is no instrumentation commercially available for measuring small amounts of oil content, and small amounts of solids in the water outlet line from a subsea located cyclone. Sample collection by an ROV (remotely operated vehicle) operated from a surface vessel, is the method to choose for an actual verification of the operation. There is a demand for a system and a method for control of a subsea located cyclone for separating oil from water, providing more accurate control and verification of the separation effect. If solids content can also be measured and mitigated, this is also in demand.

### SUMMARY OF THE INVENTION

The demand is met by the present invention providing a system for control of a subsea located cyclone for separating oil from water. The cyclone is arranged to receive water with oil contents through an inlet line, the oil is separated from the water and delivered through an oil outlet to an oil outlet line, and the water is delivered through a water outlet to a water outlet line. The system is comprising a control valve in the oil outlet or oil outlet line from the cyclone, a first differential pressure transducer arranged between the inlet line and the oil outlet from the cyclone, and a second differential pressure transducer arranged between the inlet line and the water outlet from the cyclone. The system is distinguished in that a sensor for measuring oil contents is arranged in the water outlet or water outlet line, and via a control means said sensor is operatively connected to the control valve.

Preferably, the control valve is arranged to operate according to a setpoint for the ratio between the first and second differential pressures, which setpoint and control valve opening are arranged to be adjusted as a response to a change in oil in water contents, as measured with the sensor.

Preferably the sensor is an optical “dark field” illumination sensor as described and illustrated in the parallel patent application NO 2009 3598, to which reference is made for detailed information. More specifically, this is an optical type of sensor with objective and camera arranged between a multitude of light sources, arranged outside to or including a window to be arranged in the wall of the pipe transporting the flow to be measured. Alternatively, the sensor is according to the teaching of EP 1159599. In one embodiment, the sensor is a oil in water sensor, in a more preferred embodiment the sensor is capable of determining the contents of oil and also solid particles, if any, in the water outlet flow, which is preferable because it allows preventive measures to be taken in order to prevent injection of solid particles that may have a plug effect in the reservoir. More specifically, upstream sand separation equipment like sand traps and sand separators, are set into more intense operation, or back flushed in order to improve the sand separation effect, if sand is detected in the water from the cyclone or other separation equipment. Additionally or alternatively, water containing sand can be dumped through a dump outlet upstream of a water injection pump, preferably after opening up a control valve in the oil outlet from the cyclone in order to have cleaner water in the water outlet form the cyclone, preferably sufficiently clean water to allow dumping without breaking any regulations. Unprocessed or filtered seawater may be injected until the water in the cyclone outlet line has been verified to be clean enough for safe injection, as verified by operating the sensor.

The invention also provides a method for control of a subsea located cyclone for separating oil from water. The cyclone is arranged to receive water with possible oil contents through an inlet line, the oil is separated from the water and delivered through an oil outlet to an oil outlet line, and the water is delivered through a water outlet to a water outlet line, a control valve is arranged in the oil outlet or oil outlet line from the cyclone, a first differential pressure transducer is arranged between the inlet line and the oil outlet from the cyclone, and a second differential pressure transducer is arranged between the inlet line and the water outlet from the cyclone. The method is distinguished in that an oil contents sensor is arranged in the water outlet or water outlet line, and the control valve is operated according to a setpoint for the ratio between the first and second differential pressures, which setpoint and control valve opening are adjusted as a response to a change in oil in water contents, as measured with the sensor.

Preferably a PID-controller maintains a ratio of the first differential pressure to the second differential pressure at a constant value, by controlling the control valve in the oil outlet or oil outlet line. If the oil in water contents, as measured with the sensor, exceeds a limit, the differential pressure ratio is preferably increased, whereby the control valve opens more and more oil is separated from the water.

The invention also provides use of a darkfield sensor for measuring at least one of oil contents and solids contents in a flow of water in a pipe to or from a subsea located separation equipment. Preferably the darkfield sensor is arranged with objective and camera between or encompassed by light sources, such as outside to or including a window to be arranged in a pipe wall in a pipe to or from a subsea separation equipment such as a subsea located hydrocyclone, for measuring at least one of oil contents and solids contents in a flow of water, for providing useful information for control of the subsea separation equipment.

### FIGURES

The invention is illustrated with one FIGURE, namely

FIG. 1 illustrating an embodiment of a system of the present invention.

### DETAILED DESCRIPTION

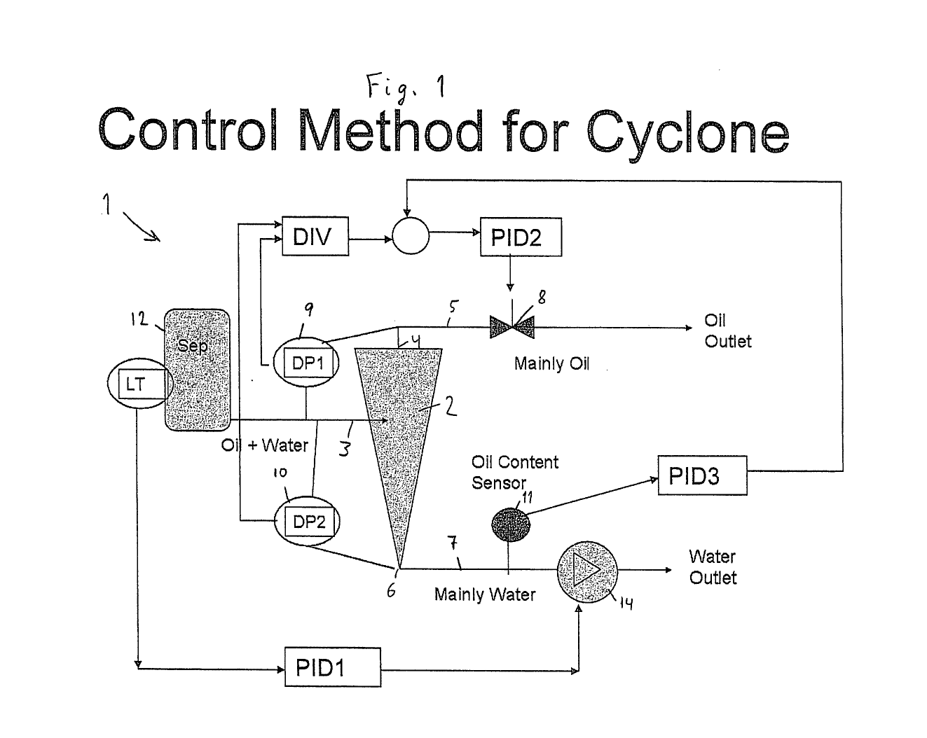

Reference is made to FIG. 1 illustrating a system of the present invention and some adjacent parts. A system 1 for control of a subsea located cyclone 2 for separating oil from water is illustrated. The cyclone 2 is arranged to receive water with oil contents through an inlet line 3, the oil is separated from the water and delivered through an oil outlet 4 to an oil outlet line 5, and the water is delivered through a water outlet 6 to a water outlet line 7. The system comprises a control valve 8 in the oil outlet or oil outlet line from the cyclone, a first differential pressure transducer 9 arranged between the inlet line and the oil outlet from the cyclone, a second differential pressure transducer 10 arranged between the inlet line and the water outlet from the cyclone and a sensor 11 for measuring oil contents is arranged in the water outlet or water outlet line, and via a control means PID3 said sensor is operatively connected to the control valve 8, via a controller PID2. Also said differential pressure transducers, or the ratio between them, DIV, are operatively connected to the control valve 8, via the controller PID2. Accordingly, the differential pressure between inlet and oil outlet is measured via sensor DPT1, the differential pressure between inlet and water outlet is measured via sensor DPT2. The computational element DIV calculates the ratio of these two signals and feeds this as a “measured Value” to a Proportional/Integrating/Derivative (PID) controller PID2.

As long as the oil fluid properties are constant, the droplet size distribution is constant, and the cyclone inlet oil concentration is constant, maintaining the ratio of DP1 to DP2 constant provides a defined split of the inlet flow between the two outlets. If the pump (14) speed is varied in response to a separator (12) level change, the control valve (8) will then change it's position such that the flow rations out of the cyclone have a constant ratio.

If the droplet size distribution changes such that the average droplet size decreases, then the separation efficiency of the cyclone also decreases. Droplets with half the size separate at appr. ⅛ of the speed. Smaller droplets coming into the cyclone will thus lead to an increasing amount of oil in the water outlet. This would be detected by the oil content sensor (11), and via the controller PID3 the ratio setpoint to the controller PID2 would be adjusted, such that a larger proportion of the incoming fluid is sent via the oil outlet through control valve (8). This will decrease the oil content in the water outlet, at the cost of increasing the water content in the oil outlet. The setpoint to the controller PID3 is the desired oil concentration in the water outlet.

If the droplet size distribution changes such that the average droplet size increases, then the separation efficiency of the cyclone also increases. Droplets with twice the size separate at appr. 8 times the speed. Larger droplets coming into the cyclone will thus lead to an decreasing amount of oil in the water outlet. This would be detected by the oil content sensor (11), and via the controller PID3 the ratio setpoint to the controller PID2 would be adjusted, such that a smaller proportion of the incoming fluid is sent via the oil outlet through control valve (8). This will increase the oil content in the water outlet, at the same time decreasing the water content in the oil outlet.

The objective of the controller PID3 is thus to maintain the oil content in the water outlet at a certain setpoint, thus at the same time minimising the water content in the oil outlet from the cyclone.

Typically there is a separator vessel (12) upstream the cyclone, as illustrated on FIG. 1, e.g. separating water from oil. The oil/water interface level is sensed by the level sensor LT in FIG. 1, and this level signal is sent to a level controller PID1. PID1 has a setpoint for the desired interface level, and may adjusts the speed of a water injection pump (14), also illustrated on FIG. 1, such that the level is controlled around it's setpoint. Accordingly, a rising level in the separator, as transmitted by a level transmitter LT, results in a pump speed increase, and vice versa.

The flow split or separation effect of the cyclone is controlled by the control valve 8, whereby the PID2 controller maintains a ratio of the first differential pressure 9 (DP1) to the second differential pressure 10 (DP2), as indicated by DIV on FIG. 1. Accordingly, the inlet flow is split in a certain ratio between the two outlets. However, the flow split setpoint to controller PID2 is calculated and adjusted within an acceptable range by the controller PID3, the range representing an acceptable operating range of the cyclone. More specifically, if the oil content in the water outlet increases, as measured by the sensor 11, controller PID3 will adjust the flow split setpoint such that more fluid is sent to the oil outlet, i.e. the control valve is opened somewhat. If the oil content in the water decreases, as measured by the sensor, controller PID3 will adjust the flow split setpoint such that less fluid is sent to the oil outlet, i.e. the control valve is closed somewhat. If the inlet flow composition varies the setpoint and thereby the separation effect of the cyclone, will be adjusted accordingly in order to ensure a consistent composition of the outlet flows from the cyclone.

The invention also comprises a system for control of subsea located separation equipment for separating water from other fluids such as oil and gas, the equipment is arranged to receive fluid with water contents through an inlet line, the water is separated from the other fluids and the water is delivered through a water outlet to a water outlet line, the other fluids are delivered through at least one fluid outlet to at least one further fluid outlet line, and the system is comprising at least one control device for control of the water separation effect, distinguished in that a sensor for measuring oil contents, and preferably also solid particle contents, is arranged in the water outlet or water outlet line, and via a control means said sensor is operatively connected to the control device. This system comprises any subsea located separation units, equipment or packs, for which the sensor can verify that the water separation effect is as intended. The sensor can also verify that the separated water is clean enough for dumping or injection. The other fluids can for example be transported further through oil lines, gas lines or multiphase fluid lines, or be subject to further processing.

The invention also comprises a method for control of subsea located separation equipment for separating water from other fluids such as oil and gas, the equipment is arranged to receive fluid with water contents through an inlet line, the water is separated from the other fluids and the water is delivered through a water outlet to a water outlet line, the other fluids are delivered through at least one fluid outlet to at least one further fluid outlet line, and the system is comprising at least one control device for control of the water separation effect, distinguished in that a sensor for measuring oil contents, and preferably also solid particle contents, is arranged in the water outlet or water outlet line, said sensor is operatively connected to the control device, whereby input from the sensor is used to control the separation effect and verify the contents of other fluids, and preferably also solid particles, in the separated water. This method is useful for any subsea located separation units, equipment or packs, for which the sensor can verify that the water separation effect is as intended. The sensor can also verify that the separated water is clean enough for dumping or injection. The other fluids can for example be transported further through oil lines, gas lines or multiphase fluid lines, or be subject to further processing.

The invention also comprises a system and a method where the sensor is arranged in a gas outlet from subsea located separation equipment, the sensor is operatively connected to means to control the separation effect, and the sensor is thereby used to control and verify the separation effect.

The systems of the invention can be combined with features as described or illustrated in this document in any operative combination, which combinations are embodiments of the present invention. The methods of the invention can be combined with features as described or illustrated in this document in any operative combination, which combinations are embodiments of the present invention.

1. A system for control of a subsea located cyclone for separating oil from water, the cyclone being arranged to receive water with oil contents through an inlet line, the oil being separated from the water and delivered through an oil outlet to an oil outlet line, and the water is delivered through a water outlet to a water outlet line, the system comprising:
a control valve in the oil outlet or oil outlet line from the cyclone;
a first differential pressure transducer arranged between the inlet line and the oil outlet from the cyclone;
a second differential pressure transducer arranged between the inlet line and the water outlet from the cyclone;
wherein a sensor for measuring oil contents is arranged in the water outlet or the water outlet line; and
wherein said sensor is operatively connected to the control valve via a control means.

2. The system according to claim 1, wherein the control valve is arranged to operate according to a setpoint for the ratio between the first and second differential pressures, which setpoint and control valve opening are arranged to be adjusted as a response to a change in oil in water contents, as measured with the sensor.

3. The system according to claim 1, wherein the sensor is a darkfield sensor with objective and camera arranged between two light sources, arranged outside to or including a window to be arranged in the pipe wall.

4. A method for control of a subsea located cyclone for separating oil from water, the cyclone being arranged to receive water with possible oil contents through an inlet line, the oil being separated from the water and delivered through an oil outlet to an oil outlet line, and the water is delivered through a water outlet to a water outlet line, a control valve is arranged in the oil outlet or oil outlet line from the cyclone, a first differential pressure transducer is arranged between the inlet line and the oil outlet from the cyclone, and a second differential pressure transducer is arranged between the inlet line and the water outlet from the cyclone, wherein an oil contents sensor is arranged in the water outlet or water outlet line, and the control valve is operated according to a setpoint for a ratio between the first and second differential pressures, which setpoint and control valve opening are adjusted as a response to a change in oil in water contents, as measured with the sensor.

5. The method according to claim 4, wherein a PID-controller maintains a ratio of the first differential pressure to the second differential pressure at a constant value, by controlling the control valve in the oil outlet or oil outlet line.

6. The method according to claim 5, wherein if the oil in water contents, as measured with the sensor, exceeds a limit, the differential pressure ratio is increased, whereby the control valve opens more and more oil is separated from the water.

7. A system for control of subsea located separation equipment for separating water from other fluids such as oil and gas, the subsea located separation equipment is arranged to receive fluid with water contents through an inlet line, the water being separated from the other fluids and the water is delivered through a water outlet to a water outlet line, the other fluids are delivered through at least one fluid outlet to at least one further fluid outlet line, the system comprising:
at least one control device for control of the water separation effect;
a sensor for measuring oil contents is arranged in the water outlet or water outlet line; and
wherein said sensor is operatively connected to the control device via a control means.

8. A method for control of subsea located separation equipment for separating water from other fluids such as oil and gas, the subsea located separation equipment being arranged to receive fluid with water contents through an inlet line, the water being separated from the other fluids and the water is delivered through a water outlet to a water outlet line, the other fluids are delivered through at least one fluid outlet to at least one further fluid outlet line, the system comprising:
at least one control device for control of the water separation effect;
wherein a sensor for measuring oil contents is arranged in the water outlet or water outlet line;
wherein said sensor is operatively connected to the control device; and
wherein input from the sensor is used to control the separation effect and verify the contents of other fluids and solid particles in the separated water.

9. The method according to claim 8, wherein upstream separation equipment like sand traps and sand separators are set into more intense operation, or back flushed in order to improve the sand separation effect, if sand is detected in the water from the separation equipment.

10. Use of a darkfield sensor for measuring at least one of oil contents and solid contents in a flow of water in a pipe to or from a subsea located separation equipment.
